(19) United States
(12) Patent Application Publication
(10) Pub. No.: US 2004/0180077 A1
(43) Pub. Date: Sep. 16, 2004

(54) Rapidly dissolving edible strips for treating obesity

(76) Inventors: Donald K. Riker, Chattanooga, TN (US); Joseph Czerwinski, Chattanooga, TN (US); Tom Knopp, Chattanooga, TN (US)

Correspondence Address: DOUGLAS T. JOHNSON, MILLER & MARTIN, 1000 VOLUNTEER BUILDING, 832 GEORGIA AVENUE, CHATTANOOGA, TN, 37402-2289, US

(21) Appl. No.: 10/794,829

(22) Filed: Mar. 5, 2004

Related U.S. Application Data
(60) Provisional application No. 60/452,592, filed on Mar. 5, 2003.

(51) Int. Cl. A61K 31/522; A61K 35/78; A61K 47/00

(52) U.S. Cl. 424/439; 424/728; 424/729; 514/263.31

(57) ABSTRACT

This invention relates to the administration of ingredients which reduce the appetite, promote weight loss and increase energy using rapidly dissolving films or strips. The benefits of the invention may be achieved through a variety of different mechanisms including addition of active ingredients, including ingredients which alter taste, promote satiety and boost energy, to the rapidly dissolving strips.

### FIELD OF THE INVENTION

The present invention relates to a rapidly dissolving film or strip for human consumption consisting of active ingredients, flavorings, colorings, or excipients that rapidly dissolve in the buccal-oral cavity. The film or strip may be absorbed and act systematically, may act locally, or provide the user with a sensory experience in order to reduce the appetite, increase metabolism, alter taste or prevent eating. Such action would result in reduced body weight, reduced interest in high-caloric or fatty food items, reduced desire to eat, and/or foster healthier eating, or foster satiety, or impart energy.

### BACKGROUND OF THE INVENTION

In recent years, edible films or strips have been used as a vehicle for inconspicuously administering a variety of agents including pharmaceutically active agents. See for example, JP 5-236885, which teaches edible films for inconspicuously administering breath-freshening agents. These products can include a variety of ingredients for the application and release of active substances in the buccal cavity.

U.S. Pat. No. 5,518,902 to Ozaki et al. discloses high pullulan content products, in the form of edible films, pharmaceuticals and others. The products can include a variety of ingredients in addition to pullulan, such as other polysaccharides, polyhydric alcohols, antiseptics, flavor-imparting agents, therapeutic agents and biologically active agents.

U.S. Pat. No. 5,411,945 to Ozaki et al. discloses a binder, and products produced therewith, such as edible films, which comprise pullulan and saccharides. The products can include a variety of ingredients in addition to pullulan, such as other polysaccharides, antibacterial agents, flavor-imparting agents and pharmaceutically and biologically active substances.

U.S. Pat. No. 4,851,394 to Kubodera discloses edible films made from glucomannan and polyhydric alcohol. The films may be eaten as such or used as edible food packages. Alternatively, the film may also serve as a high-performance filter medium or a wound dressing. Additionally, the edible films may include other natural polysaccharides, an alkali, and food materials.

U.S. Pat. No. 3,784,390 Hijiya et al. discloses pullulan edible films for providing packing materials for food, pharmaceuticals and other oxygen sensitive materials.

U.S. Pat. No. 4,623,394 Nakamura et al. discloses a gradually disintegrable molded article that can be a film made with pullulan. The molded article is usable for industrial materials, pharmaceuticals and consumers' products.

U.S. Pat. No. 4,562,020 Hijiya et al. discloses a process for producing a self-supporting film of a glucan, which can be pullulan.

U.S. Pat. No. 5,629,003 Horstmann et al. discloses an edible film which permits the individual dosage of drugs, confectionary, other food, cosmetics and the like.

Japanese Patent Document JP5001198 discloses films made of polyvinyl alcohol and at least one of carrageenan, water-soluble cellulose, alpha-starch or pullulan.

WO 98/20862 discloses a film for use in the oral cavity that can contain a cosmetic or pharmaceutical active substance.

WO 98/26763 discloses a flat, foil, paper or wafer like presentation for release of active substances into the buccal cavity. The particular active substance disclosed is apomorphine or one of its therapeutically suitable salts.

WO 01/70194 discloses edible films containing an ion exchange resin to mask the taste of a pharmaceutically active agent therein.

WO 00/18365 discloses edible films that include pullulan and antimicrobially effective amounts of essential oils effective against dental plaque, gingivitis and bad breath.

Despite the existence of rapidly dissolving orally consumable films in the prior art, these films have typically been utilized with either pharmaceutically active substances with special properties suited for buccal delivery, or for treatment to the buccal cavity, as breath fresheners. None of the above-listed references disclose employing a film for the administration of substances that suppress appetite or food cravings, or promote satiety, or result in weight or fat loss. Moreover, none of these references reveal substances that promote or induce energy, such as the methylxanthine compounds, including caffeine and green tea.

The inventor is not aware of any suggestion in the published art that buccal strips or films be used to administer products for treating obesity by suppressing appetite, increasing metabolism, and promoting weight and fat loss. Accordingly, an object of this invention is to provide rapidly dissolving edible strips for treating obesity, or promoting weight loss, increased energy, or satiety.

A variety of methods are currently used to administer products for treating obesity by suppressing the appetite and promoting weight and fat loss. The methods typically involve oral administration of different types and formulations of products, as well as guidelines for diet and activity. Some products are administered in the form of tablets or gelatin capsules. Other products are powders or granules suitable for preparation in an aqueous suspension by the addition of water. Tablets and gelatin capsules typically require a liquid to aid in the administration of these products. Powders and granules require the addition of water before administration. These products are obtrusive and often cause embarrassment for the consumer who wishes to inconspicuously take the products. Moreover, these products are difficult to administer because the consumer typically requires a liquid to aid in administration of the products. A more acceptable method of unobtrusively delivering these products is needed.

Most weight loss products are directed to increasing thermogenesis or caloric consumption, altering body chemistry to reduce appetite, or reducing caloric intake by either blocking caloric absorption by the body or ingesting foods or supplements that are low in calories (or not easily digested). It is desirable to provide alternative mechanisms to aid in weight loss regimens, such as no or very low calorie taste/craving satisfaction, or taste modification.

Many other weight loss products take the form of food, or food replacements. These include diet bars, power bars, nutrition bars, energy slurries, pre-mixed ready-to-drink beverages, high protein or high carbohydrate products. Despite their appellations these products generally result in a net caloric intake and may not offer metabolic savings thereby resulting in additional weight gain. It is desirable to provide the user with satiety, or energy gain, or weight loss, or appetite suppression without additional caloric intake.

The current invention consists of a product and method for inconspicuously delivering substances to promote weight loss and appetite suppression. The invention comprises a rapidly dissolving thin film, or strip, or wafer which is easily portable and small, typically 1-2 cm in width and length. This product does not require swallowing a pill or liquid, is socially acceptable, can be flavored and can be adjunctive to other treatment modalities. It will obviate or substitute for between-meal snacks and other caloric loads.

### SUMMARY OF THE INVENTION

The present invention recognizes and addresses the foregoing disadvantages, and others, of prior art compositions and methods.

Briefly, the present invention is a novel rapidly dissolving edible strip or film for treating obesity, suppressing the appetite, promoting weight loss, and increasing energy or satiety. The rapidly dissolving edible strip or film of the present invention contains active ingredients that promote weight loss, suppress the appetite and increase energy. The present invention may also include a variety of ingredients including various flavorings or sweeteners. The present invention provides an effective method for treating obesity by using an unobtrusive, portable edible strip or film that can be administered inconspicuously without the requirement of a liquid to aid in administration.

One novel aspect of the invention is the use of an unobtrusive film or strip for administration of ingredients which treat obesity, suppress the appetite, promote weight loss, and increase energy or satiety.

Another novel aspect of the invention is the use of an easily portable film or strip for administration of ingredients which treat obesity, suppress the appetite, promote weight loss, and increase energy or satiety, which does not require swallowing a pill or liquid.

Accordingly, it is an object of the present invention to provide an edible film or strip for the administration of ingredients which treat obesity by suppressing the appetite, promoting weight loss, increasing energy, and increasing satiety.

More particularly, it is an object of the present invention to provide a product for delivering ingredients which promote weight loss or increase energy which is portable, unobtrusive, and can be delivered inconspicuously without the requirement of a liquid to aid in the administration of the product.

Additional objects and advantages of the invention will be set forth in part in the following description, or may be obvious from the description, or may be learned through practice of the invention.

### DETAILED DESCRIPTION OF THE INVENTION

This invention comprises a rapidly dissolving thin strip or film for human consumption consisting of active ingredients, flavorings, colors or excipients that rapidly dissolve in the buccal-oral cavity. The strips may be made of complex carbohydrates, such as pullalan, or be starch-based. The strips may be used to reduce appetite, or the desire to eat, increase satiety, or alter taste, or prevent or inhibit eating, or provide energy. This quick dissolving system induces an immediate experience and acts to promote rapid buccal-oral absorption of the active ingredients resulting in faster bioavailability and efficacy of active ingredients.

Many active ingredients known in the art for reducing the appetite, promoting weight loss or thermogenesis may be used in connection with the edible strip. Active ingredients may include caffeine, green tea extract, theobromine, ginseng root and amino acids. Caffeine is known to promote weight loss by increasing thermogenesis and energy. Theobromine is found in products of the cocoa tree. It is found in chocolate products and used in medicine as a diuretic, vasodilator, and myocardial stimulant. It affects humans similarly to caffeine, but on a much smaller scale. Green tea has long been known to have an effect on body weight and energy expenditure. Epigallocatechin gallate (abbreviated herein as EGCG) is a naturally-occurring substance found chiefly in green tea and green tea extracts. EGCG is ideal as a weight loss agent because of its lack of toxicity or apparent side effects. EGCG and related catechins occur naturally in several types of plants, including tea, and thus have a long history of safety in that form. Ginseng root has been shown to have therapeutic value in increasing energy, reducing stress and improving memory. Amino acids such as theonine and arginine have also been found to promote weight loss. Theonine is known to have calming effects and promote a feeling of satiety. Arginine inhibits the absorption of dietary fat and is also known to combat fatigue.

Additional ingredients maybe added to the composition of the present invention, including, but not limited to anti-oxidants, vitamins, minerals and other trace elements. Additionally, artificial sweeteners, sugar alcohols and glycerol, organic acids, stabilizers and gums may also be added to the composition of the present invention. A variety of supplements may also be added to the composition. The strips maybe used in connection with any active ingredients that suppress the appetite or promote weight and fat loss. The strips may be administered before, after and between meals. Such strips could alternatively be discreetly dissolved directly into foods or drinks to provide similar benefits and actions.

The edible strips can also include natural and artificial flavors including, mint, chocolate, spice, salt, vanilla, citrus, fruit, herbs, coffee and other food flavors.

The benefits of the present invention include a product and method for suppressing the appetite and promoting weight and fat loss, which are portable, convenient, and socially acceptable. This form of delivery is novel to the appetite suppression/weight control field because it provides the user with a wholly new paradigm to manage daily, hour-to-hour appetite suppression in a convenient, portable and socially acceptable dose form.

The benefits of the present invention may be achieved through a number of variations of this invention. Variations of this invention may include, but are not limited to use of this product as a no or low calorie sweetener. For example, the edible strips may be used as a sweetener for various foods and drinks. The strips can be sweetened by combining them with sugars or low caloric sweeteners such as saccharine or aspartame to promote weight loss by reducing net caloric intake. The buccal-oral strips may be used to sweeten coffee or tea by being held in the buccal cavity while consuming these beverages.

Additionally, the invention may be used for taste alteration. For example, the oral strips may be used to distort food flavors. By combining the strip with Gymnema sylvestre, menthol, aromatics, quinine, lithium, cyclodextrins, and/or others, the buccal strips may be used to mask the taste of various foods. For example, a strip may be consumed prior to eating high caloric content foods to mask the pleasant taste of the food, or change its palatability, thereby reducing future cravings for the food. The strips may even be used to make some food flavors unpleasant, and the food therefore unpalatable.

The invention may also be used to alter taste by taste reduction when combined with ingredients such as benzocaine, capsaicin, wasabi, and local anesthetics to mask the flavor of unpleasant foods or pharmaceuticals. For example, a strip may be administered prior to consuming very spicy foods to reduce the taste of the food. The invention may also be used in connection with taste amplification by combining the strips with ingredients such as monosodium or monosodium glutamate. For example, the strips may be used prior to consuming low caloric diet foods to make them more tasty and flavorful.

The invention may also be used in place of food substitutes such as diet beverages and shakes, diet products, food items, or snacks to reduce the daily caloric intake of an individual. The strips may provide the user with a burst of flavor imparting an intense sensory experience similar to the food being craved, without the consumption of excess calories. For example, the strips may be chocolate flavored, with low or no calories. Therefore, when an individual craving chocolate consumes a strip, the individual will satisfy the craving with a net loss in daily calories consumed. By replacing a high fat or high calorie content food item with flavored strips that provide low or no calories, the user satisfies the craving thereby decreasing caloric intake. The strips may also be used as replacements for sugars, fats, salts and other flavors.

The benefits of the present invention may also be achieved by combining the oral strips with complex flavor systems that mimic or induce nutritional sensations that promote appetite or satiety. For example, ingredients that mimic or induce sensations that promote satiety may be added to the strips. Thus, when an individual senses hunger, the individual can satisfy the hunger by consuming a strip. Alternatively, an individual can consuming a strip prior to meals to suppress appetite and decrease caloric intake. A strip may also be consumed in between meals to decrease snacking and therefore promote weight loss. The strip may have little or no caloric content and will give the individual feelings of satiety which will result in decreased caloric intake and weight loss.

The invention may also be used as an energy boost to aid in the promotion of weight loss. By combining the strips with energy boosting ingredients such as sugar, caffeine or green tea, an individual can consume a low or no calorie strip that provides a boost of energy. In this way, the strips can serve as a replacement for other high caloric energy boosting foods, such as caffeinated sodas (soft drinks) having high sugar content, chocolate bars, or coffee, thereby reducing the net daily caloric intake of an individual.

This invention may also be used to promote dry mouth. By combining the strips with zinc or zinc salts, the strips may be administered to promote dry mouth by decreasing saliva production. This decreases the taste sensations making foods less flavorful and thereby reducing food consumption.

This invention may also be used to inconspicuously provide antioxidants and vitamins. The rapidly dissolving buccal strips may be combined with antioxidants and vitamins and be administered in place of tablets, capsules and powders.

In the preferred embodiment, the edible strip comprises at least about 50% by weight of the pullulan or starch based film medium and between about 10% to 50% by weight or between about 5 mg to 25 mg of one or more of caffeine, green tea extract, theobromine, Asian (Panax) ginsing root, and amino acids such as theonine and arginine.

One edible strip of the preferred embodiment according to the present invention has the following composition:

| Ingredient | % by weight prior to drying |
|---|---|
| Deionized Water | 59.6974689 |
| Gellan Gum or other gum | 0.1299030 |
| Caffeine, Green Tea Extract, | 7.9596617 |
| Theobromine, Asian (Panax) ginseng | |
| root, or amino acid | |
| Propylene Glycol | 0.5247080 |
| High Frustose Corn Syrup | 1.3881840 |
| Sorbitol (or other sugar alcohol) | 1.6097840 |
| Polysorbate 80 | 1.0468690 |
| Glycerol | 4.4900300 |
| Modified Corn Starch | 13.8765550 |
| Acesulfame K or artificial sweeteners | 2.3127920 |
| Sucralose or other artificial sweeteners | 2.3153390 |
| Flavors or organic acids | 4.6488038 |
| Dyes | 0.0001997 |
| | 100% |

The edible strip of the preferred embodiment according to the example above is produced by preparing a solution of the above-listed ingredients. The solution is mixed and heated up to 150° F. depending on the actual ingredients used. The resulting solution is them evenly coated onto a conveyer. The solvents are removed and the residual water is adjusted through a series of drying stages until the desired thickness and texture is achieved. The dried film is then rolled and may be cut to the desired size and shape.

Typical strips or films will be between 1-5 mm thick, 10-50 mm wide, 10-50 mm long and weigh 20-100 mg. In the preferred embodiment, the strips will weight about 50 mg each and have the following dimensions: 2.7 mm×25 mm×33 mm.

Packaging of individual strips or other forms will require low humidity conditions provided by packaging barriers to ambient air so as to enhance stability. To allow users to gain full benefit from this conception may require packaging of multiple individual items in a convenient, portable package.

While preferred embodiments of the invention have been described above, it is to be understood that any and all equivalent realizations of the present invention are included within the scope and spirit thereof. Thus, the embodiments depicted are presented by way of example only and are not intended as limitations upon the present invention. While particular embodiments of the invention have been described and shown, it will be understood by those skilled in the art that the present invention is not limited thereto since many modifications can be made.

We claim:

1. An edible strip comprising an effective amount of an ingredient that promotes at least one of: taste sweetening, taste alteration, appetite reduction, increased energy, dry mouth, increased metabolism, and reduced body weight.

2. An edible strip according to claim 1, wherein said ingredient is selected from the group consisting of caffeine, green tea extract, theobromine, ginseng root, theonine, arginine and mixtures thereof.

3. The edible strip according to claim 1, comprising an effective amount of caffeine.

4. The edible strip according to claim 1, comprising an effective amount of green tea extract.

5. The edible strip according to claim 1, comprising an effective amount of theobromine.

6. The edible strip according to claim 11, comprising an effective amount of ginseng root.

7. The edible strip according to claim 1, comprising an effective amount of an amino acid, selected from the group consisting of theonine, arginine and mixtures thereof.

8. An edible strip for increasing energy or satiety, comprising an effective amount of caffeine.

9. The edible strip according to claim 8, further comprising an effective amount of green tea extract.

10. The edible strip according to claim 8, further comprising an effective amount of theobromine.

11. The edible strip according to claim 8, further comprising an effective amount of ginseng root.

12. The edible strip according to claim 8, further comprising an effective amount of an amino acid, selected from the group consisting of theonine, arginine and mixtures thereof.

13. An edible strip for the treatment of obesity, comprising an effective amount of an agent that promotes thermogenesis.

14. The edible strip according to claim 13, wherein the agent for promoting thermogenesis is selected from the group consisting of caffeine, green tea extract, theobromine, ginseng root, theonine, arginine and mixtures thereof.

15. The edible strip according to claim 1, comprising between about 10% to about 50% by weight caffeine.

16. The edible strip according to claim 8, comprising between about 10% to about 50% by weight caffeine.

17. The edible strip according to claim 1, comprising up to 100 mg caffeine.

18. The edible strip according to claim 8, comprising up to 100 mg caffeine.

17. A method for the treatment of obesity or inducing weight loss in human subjects comprising administration of an edible strip.

18. The method according to claim 17, wherein the edible strip contains an effective amount of at least one of: caffeine, green tea, theobromine, ginseng root, theonine, and arginine.

18. The method according to claim 17, wherein the edible strip comprises between about 10% to about 50% by weight caffeine.

19. The method according to claim 17, wherein the edible strip comprises between about 10% to about 50% by weight green tea extract.

20. The method according to claim 17, wherein the edible strip comprises between about 10% to about 50% by weight theobromine.

21. The method according to claim 17, wherein the edible strip comprises between about 10% to about 50% by weight ginseng root.

22. The method according to claim 17, wherein the edible strip comprises between about 10% to about 50% by weight of amino acid, selected from the group consisting of theonine, arginine and mixtures thereof.
